(19) United States
(12) Patent Application Publication
(10) Pub. No.: US 2025/0228972 A1
(43) Pub. Date: Jul. 17, 2025

(54) METHOD OF PRODUCTION AND USE OF PREPARATION COMPRISING CODON-OPTIMIZED HUMAN COAGULATION FACTOR IX (hFIXco)-TRANSDUCED HUMAN UMBILICAL CORD MESENCHYMAL STEM CELLS (tr-HUCMSCs)

(71) Applicant: CHILDREN'S HOSPITAL OF ZHEJIANG UNIVERSITY SCHOOL OF MEDICINE, HANGZHOU CITY (CN)

(72) Inventors: YONGMIN TANG, HANGZHOU CITY (CN); ZIBIN BU, HANGZHOU CITY (CN); XIAOJUN XU, HANGZHOU CITY (CN); WEIQUN XU, HANGZHOU CITY (CN)

(21) Appl. No.: 18/974,277

(22) Filed: Dec. 9, 2024

(30) Foreign Application Priority Data
Jan. 15, 2024 (CN) 202410056025.7

(51) Int. Cl. A61K 48/00; A61K 35/51; A61K 38/36; A61P 7/04; C12N 15/86

(52) U.S. Cl. CPC A61K 48/005; A61K 35/51; A61K 38/36; A61P 7/04; C12N 15/86; C12N 2750/14143; C12N 2750/14151; C12N 2800/22

(57) ABSTRACT

A method for producing a preparation containing codon-optimized human coagulation factor IX (hFIXco)-transduced human umbilical cord mesenchymal stem cell (tr-HUCMSCs) is provided. The preparation method includes the following steps: step 1, subjecting an F9 gene to codon-optimization to obtain a hFIXco gene, loading the hFIXco gene into ScAAV-DJ/8, and then adding a human apolipo-protein hepatic control region and a human α1-antitrypsin gene promoter in front of the hFIXco gene to form a double-stranded adeno-associated virus (AAV) vector ScAAV-DJ/8-LP1-hFIXco with stably high expression and a hFIXco activity; and step 2, transducing the ScAAV-DJ/8-LP1-hFIXco into HUCMSCs, mixing the ScAAV-DJ/8-LP1-hFIXco with digested HUCMSCs at a ratio of 1,000:1 to allow culture, and then collecting the cells at 24 h after the mixing to obtain a preparation containing tr-HUCMSCs.

### CROSS REFERENCE TO RELATED APPLICATION

This patent application claims the benefit and priority of Chinese Patent Application No. 202410056025.7 filed with the China National Intellectual Property Administration on Jan. 15, 2024, the disclosure of which is incorporated by reference herein in its entirety as part of the present application.

### TECHNICAL FIELD

The present disclosure relates to the technical field of stem cell preparations, and in particular to a method of production and use of a preparation comprising codon-optimized human factor IX (hFIXco)-transduced human umbilical cord mesenchymal stem cells (tr-HUCMSCs).

### BACKGROUND

Hemophilia B is an X-linked recessive monogenic disease caused by mutations in the F9 gene. This leads to a deficiency or dysfunction of coagulation factor IX (FIX) in plasma, resulting in abnormal coagulation function. Hemophilia B accounts for 15% to 20% of all hemophilia, with an incidence rate of approximately 1/25,000. Currently, hemophilia B is mainly treated with lifelong prophylactic replacement therapy using recombinant FIX, once every 1 to 2 weeks. The treatment is complicated and expensive. Despite the low probability of patients developing inhibitors following recombinant FIX administration (approximately 1% to 5% of patients), re-exposure to recombinant FIX in patients with anti-FIX inhibitors may induce allergic reactions, which restrict its clinical application.

Gene therapy using adeno-associated virus (AAV) vectors holds the promise to completely cure hemophilia B. The construction of a recombinant adeno-associated virus vector (rAAV) usually involves the deletion of rep and cap genes and the insertion of a gene of interest between inverted terminal repeats (ITRs). A resulting vector plasmid is then co-transfected into tissue cells along with a packaging plasmid (which expresses the AAV rep and cap genes). Therefore, the rAAV vector can only provide therapeutic proteins that can be persistently expressed in host cells. This vector shows significant safety compared to lentiviral and retroviral vectors. Slight differences in a capsid coding sequence can significantly affect the vector tropism for tissues and improve the efficiency of gene transduction. Many naturally occurring and genetically engineered AAV capsids have been identified at present. The rAAV vector exhibits a high tropism for the liver and can effectively transduce late mitotic cells through targeted gene transfer. This reduces the risk of germline transmission and minimizes the inflammatory response. The disadvantage is that only smaller therapeutic genes can be accommodated. In general, the rAAV vectors are relatively effective and safe, making them the most suitable candidates for clinical gene therapy and the first viral vectors to be commercialized. However, AAV vectors commonly used for effective gene therapy require high dosages (ranging from 10¹¹ to 10¹² vector genomes (vg)/kg), which may cause hepatic toxicity and immune responses, ultimately restricting the widespread application of the AAV vectors.

In November 2022, the Food and Drug Administration (FDA) in US approved the first gene therapy for adult patients with hemophilia B, using AAV5 as a vector to deliver padua 1 (a FIX variant). However, efficient transduction by AAV is hampered by the need to convert its single-stranded (ss) genome into a double-stranded (ds) form that can be transcribed in target cells. AAV-DJ is a chimera of AAV types 2, 8, and 9, while AAV-DJ/8 is a mutant of the AAV-DJ in the heparin-binding domain. The AAV-DJ/8 differs from its closest natural relative (AAV-2) by 60 capsid amino acids. A recombinant AAV-DJ/8 vector outperformed eight standard AAV serotypes in cell culture and significantly outperformed AAV-2 in the liver. The double-stranded AAV-DJ/8 vector has not yet been used in gene therapy for hemophilia B.

Human umbilical cord mesenchymal stem cells (HUCMSCs) are stromal cells with self-renewal and multilineage differentiation potential. HUCMSCs serve as a desirable carrier for cell therapy due to low immunity and immunomodulatory effects.

Mesenchymal stem cells (MSCs) are currently used in the study of disease models such as cancer, diabetes, liver fibrosis, and myocardial infarction. However, there are no reports on the HUCMSCs as carrier cells for the treatment of hemophilia B.

### SUMMARY

### 1. Technical Problem to be Solved

An objective of the present disclosure is to solve the problem that HUCMSCs that can automatically secrete FIX have not been used as carrier cells for the treatment of hemophilia B in the prior art. The present disclosure provides a method of production and use of a preparation comprising codon-optimized human coagulation factor IX(hFIXco)-transduced HUCMSCs (tr-HUCMSCs).

### 2. Technical Solutions

To achieve the above objective, the present disclosure adopts the following technical solutions:

The present disclosure provides a preparation comprising codon-optimized human coagulation factor IX (hFIXco)-transduced HUCMSCs (tr-HUCMSCs).

The present disclosure further provides a method for producing the preparation comprising tr-HUCMSCs, including the following steps:
step 1, subjecting an F9 gene to codon-optimization to obtain a hFIXco gene, loading the hFIXco gene into ScAAV-DJ/8, and then adding a human apolipo-protein hepatic control region and a human α1-antitrypsin gene promoter in front of the hFIXco gene to form a double-stranded adeno-associated virus (AAV) vector ScAAV-DJ/8-LP1-hFIXco with stably high expression and a hFIXco activity; and
step 2, transducing the ScAAV-DJ/8-LP1-hFIXco into HUCMSCs, mixing the ScAAV-DJ/8-LP1-hFIXco with digested HUCMSCs at a ratio of 1,000:1 to allow culture, and then collecting cells at 24 h after the mixing to obtain a preparation comprising tr-HUCMSCs.

In some embodiments, provision of vector ScAAV8 and synthesis of ScAAV-LP1-hFIXco in step 1 are entrusted to commercial companies, and the double-stranded AAV vector ScAAV-DJ/8-LP1-hFIXco has been deposited in the China Center for Type Culture Collection (CCTCC) on Dec. 5, 2023, with a deposit number of CCTCC NO: V2023112.

In some embodiments, the ScAAV-DJ/8-LP1-hFIXco in step 1 is assembled and amplified using an AAV-DJ/8 Helper Free Bicistronic expression system kit.

In some embodiments, amplificable vectors are derived from human embryonic kidney 293 cells transformed with simian virus 40 large T antigen (HEK293T).

The present disclosure further provides use of the preparation comprising tr-HUCMSCs in treatment of human hemophilia B.

The present disclosure further provides use of the preparation comprising tr-HUCMSCs in in vivo gene therapy of a patient with FIX deficiency.

### 3. Beneficial Effects

Compared with the prior art, embodiments of the present disclosure have the following advantages.

(1) In the present disclosure, the method of production overcomes disadvantages of the traditional gene therapy that high doses of AAV vectors (approximately 10¹¹ to 10¹² vector genomes/kg) are directly injected into the body, causing bio-distribution to different unnecessary tissues randomly, which might result in unexpected side-effects. The method of production also avoids the failure of gene therapy caused by the presence of anti-AAV antibodies in vivo. The gene therapy is combined with a cell therapy to generate a cell therapy-based gene therapy. This process is a beneficial exploration for both the gene therapy and the cell therapy.

(2) In the present disclosure, a vector ScAAV-DJ/8-LP1-hFIXco is successfully introduced into the HUCMSCs for the first time, and stable expression of hFIXco has been detected at the cellular level. Significant improvement in coagulation function of mice has been observed in experimental studies using F9 gene knockout mice. This process is expected to be further applied in cell therapy-based gene therapy for hemophilia B.

### BRIEF DESCRIPTION OF THE DRAWINGS

FIGS. 1A-1B show the gene structure of the hFIXco and a schematic diagram for construction of the ScAAV-DJ/8-LP1-hFIXco according to the present disclosure;

FIG. 2 shows determination of hFIXco activities 24 h after the ScAAV-DJ/8-LP1-hFIXco according to the present disclosure is transduced into HUCMSCs (tr-HUCMSCs), normal liver cells HL7702 (tr-HL7702), Chinese hamster ovary (CHO) cells (tr-CHO), and cells of epithelial cell strain (FL) from human amniotic membrane (tr-FL);

FIG. 3 shows determination of hFIXco activities at different time points after the ScAAV-DJ/8-LP1-hFIXco according to the present disclosure is transduced into the HUCMSCs (tr-HUCMSCs) and normal liver cells HL7702 (tr-HL7702);

FIG. 4 shows determination of RNA at different time points after the ScAAV-DJ/8-LP1-hFIXco according to the present disclosure is transduced into the HUCMSCs (tr-HUCMSCs) and normal liver cells HL7702 (tr-HL7702);

FIG. 5 shows determination of hFIXco protein expression at different time points after the ScAAV-DJ/8-LP1-hFIXco according to the present disclosure is transduced into the HUCMSCs (tr-HUCMSCs);

FIG. 6 shows long-term expression observation of the hFIXco after the ScAAV-DJ/8-LP1-hFIXco according to the present disclosure is transduced into the HUCMSCs (tr-HUCMSCs) and after the ScAAV-DJ/8-LP1-hFIXco is directly injected into the tail vein of NSG mice;

FIGS. 7A-7B show measurement of coagulation time for F9 gene knockout mice treated with the ScAAV-DJ/8-LP1-hFIXco and the tr-HUCMSCs according to the present disclosure; and

FIG. 8 shows H&E staining results of liver, spleen, brain, and lung tissues in NSG mice 7 months after tail vein injection of HUCMSCs, tr-HUCMSCs, ScAAV-DJ/8-LP1-hFIXco, and 0.9% sodium chloride in the present disclosure.

### DETAILED DESCRIPTION OF THE EMBODIMENTS

The embodiments of the present disclosure are clearly and completely described below with reference to the drawings in the examples of the present disclosure. Apparently, the described examples are merely a part of rather than representing all of the embodiments of the present disclosure.

### Example 1

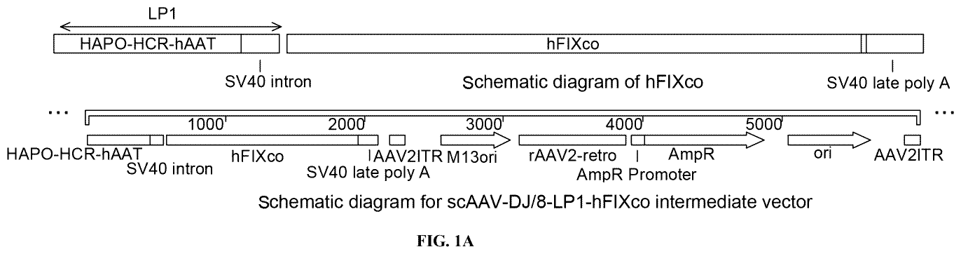

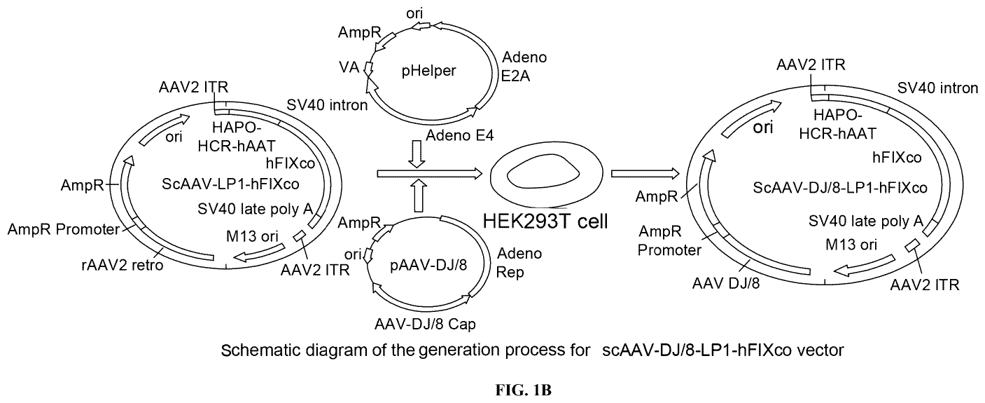

Referring to FIGS. 1A-1B, construction of an AAV expression vector ScAAV-LP1-hFIXco of the F9 gene (NCBI accession number: BC109215.1) involved the following steps. A hFIXco gene was designed and loaded into ScAAV8, and a human apolipo-protein hepatic control region (HCR) and a human α1-antitrypsin (hAAT) gene promoter was added in front of the hFIXco gene to form ScAAV-LP1-hFIXco with stable and high expression of hFIXco activity. The provision of ScAAV8 and the synthesis of ScAAV-LP1-hFIXco were both entrusted to relevant biological gene companies. Based on sequencing and alignment, the gene sequence was completely consistent with the expected designed gene sequence. The ScAAV-LP1-hFIXco was assembled in HEK293T cells under the joint action of pAAV-DJ/8 and pHelper to obtain ScAAV-DJ/8-LP1-hFIXco.

In this example, the preparation method overcame disadvantages of the traditional gene therapy that relatively high doses of AAV vectors (approximately 10¹¹ to 10¹² vg/kg) were directly injected into the body, causing bio-distribution to different unnecessary tissues randomly, which might result in unexpected side-effects. The preparation method also avoided the failure of gene therapy caused by the presence of anti-AAV antibodies in vivo. The gene therapy was combined with a cell therapy to generate a cell-based gene therapy. This process was a beneficial exploration for both the gene therapy and the cell therapy.

In this example, the ScAAV-DJ/8-LP1-hFIXco was successfully introduced into the HUCMSCs for the first time, and stable expression of hFIXco was detected at the cellular level. Significantly improvements in coagulation function of mice in experimental studies using F9 knockout mice. This process was expected to be further applied in cell-based gene therapy for hemophilia B.

### Example 2

Titration of the virus titer of ScAAV-DJ/8-LP1-hFIXco was performed as follows. Assembly and amplification of the ScAAV-DJ/8-LP1-hFIXco were completed according to the instructions of an AAV-DJ/8 Helper Free Bicistronic expression system (IRES-GFP) kit. Amplificable vectors were derived from a HEK293T cell line. The cell supernatant was collected and concentrated with a virus concentrator, and the titer of the collected ScAAV-DJ/8-LP1-hFIXco was determined according to the instructions of a Quick Titer™ AAV Quantification kit. The results showed that the virus titer was 10¹⁴ to 10¹⁵ vector genome (vg)/mL.

### Example 3

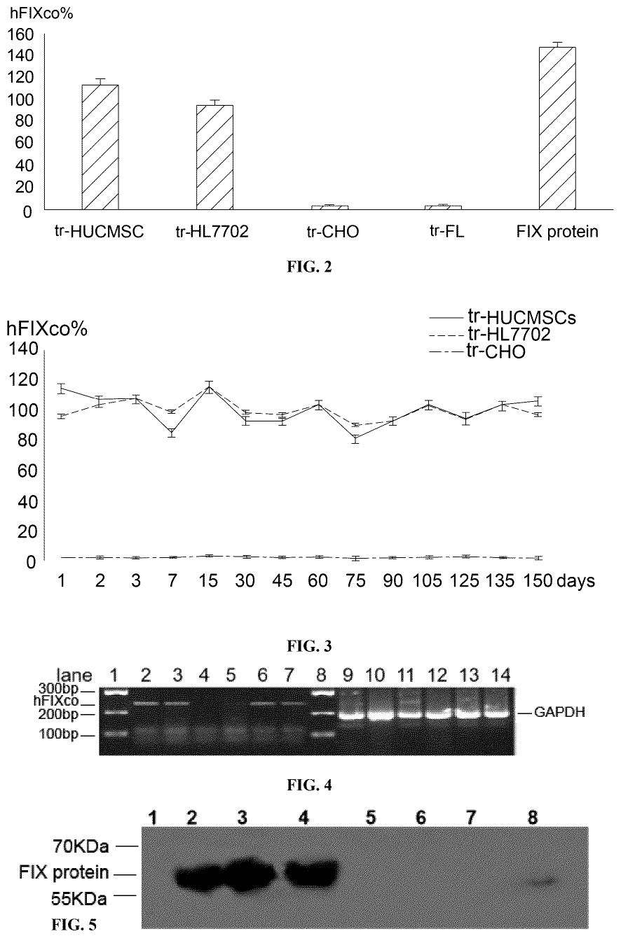

Referring to FIG. 2 to FIG. 3, the determination of hFIXco expression and activity in different host cells transduced with ScAAV-DJ/8-LP1-hFIXco involved the following steps. The ScAAV-DJ/8-LP1-hFIXco was mixed with digested HUCMSCs at a ratio of 1,000:1 to allow culture. The cells were collected 24 h, 48 h, and 72 h after the mixing, while a CHO cell (Chinese hamster ovary cell line) group, an FL (human amniotic membrane cell line) cell group, and an HL7702 (human liver cell line) cell group were set up. The results showed that only the HUCMSCs group and the HL7702 cell group could secrete active hFIXco, while the active hFIXco secreted by the CHO cell group and the FL cell group was almost undetectable. Subsequently, the hFIXco activity was measured at different time points during continuous in vitro culture for 5 months. The results showed that both the HUCMSCs group and the HL7702 cell group could stably secrete hFIXco, with a hFIXco activity of the HUCMSCs group being around 80.5% to 114.1%, while that of the HL7702 cell group being around 89.5% to 115.3%.

### Example 4

Referring to FIG. 4, the expression of hFIXco RNA of host cells transduced with ScAAV-DJ/8-LP1-hFIXco was determined as follows. HUCMSCs and HL7702 cells transduced with ScAAV-DJ/8-LP1-hFIXco were collected. The RNA was extracted, and reverse-transcribed into cDNA. Polymerase chain reaction (PCR) amplification was performed and resulting products were subjected to agarose electrophoresis, where there appeared a target band at the position of approximately 243 bp, corresponding to the expected band.

Lane 1: Marker; Lane 2: tr-HUCMSCs after 24 h; Lane 3: tr-HUCMSCs after 5 months; Lane 4: HUCMSCs not transduced with ScAAV-DJ/8-LP1-hFIXco (untr-HUCMSCs) after 24 h; Lane 5: untr-HUCMSCs after 5 months; Lane 6: HL7702 cells transduced with ScAAV-DJ/8-LP1-hFIXco (tr-HL7702) after 24 h; Lane 7: tr-HL7702 cells after 5 months; Lane 8: Marker; Lane 9 to Lane 14: GAPDH, internal reference of the above samples.

### Example 5

Referring to FIG. 5, the expression of hFIXco protein in HUCMSCs transduced with ScAAV-DJ/8-LP1-hFIXco was performed as follows. Western blotting was conducted to develop the hFIXco protein on the film, and a positive band appeared at the expected position (approximately 56 kDa), confirming that relatively satisfactory hFIXco expression could be achieved at a protein level.

Lane 1: Marker; Lane 2: Cell supernatant from tr-HUCMSCs after 24 hours transduction; Lane 3: Cell supernatant from tr-HUCMSCs after 5 months transduction; Lane 4: Cell lysate from tr-HUCMSCs after 5 months transduction; Lane 5: Cell supernatant from untr-HUCMSCs; Lane 6: Cell lysate from untr-HUCMSCs; Lane 7: Blank culture medium; Lane 8: Purified FIX protein at 8 ng/lane.

### Example 6

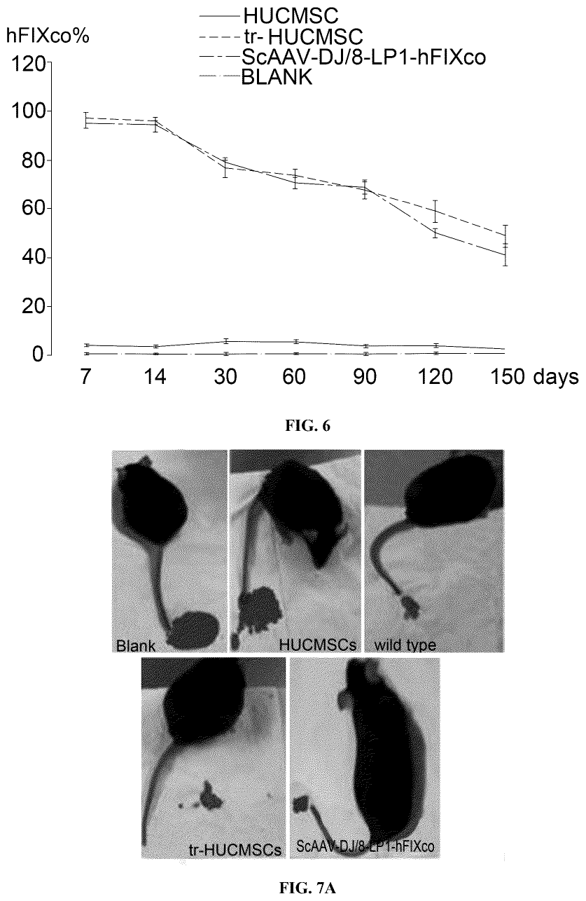

Referring to FIG. 6, the long-term expression of hFIXco following the injection of NSG mice via the tail vein with tr-HUCMSCs was performed as followings. Human FIX activity was detected 1 week, 1 month, 2 months, 3 months, 4 months, and 5 months after the injection of NSG mice via the tail vein with tr-HUCMSCs. Although the activities decreased, they were all within an acceptable range.

### Example 7

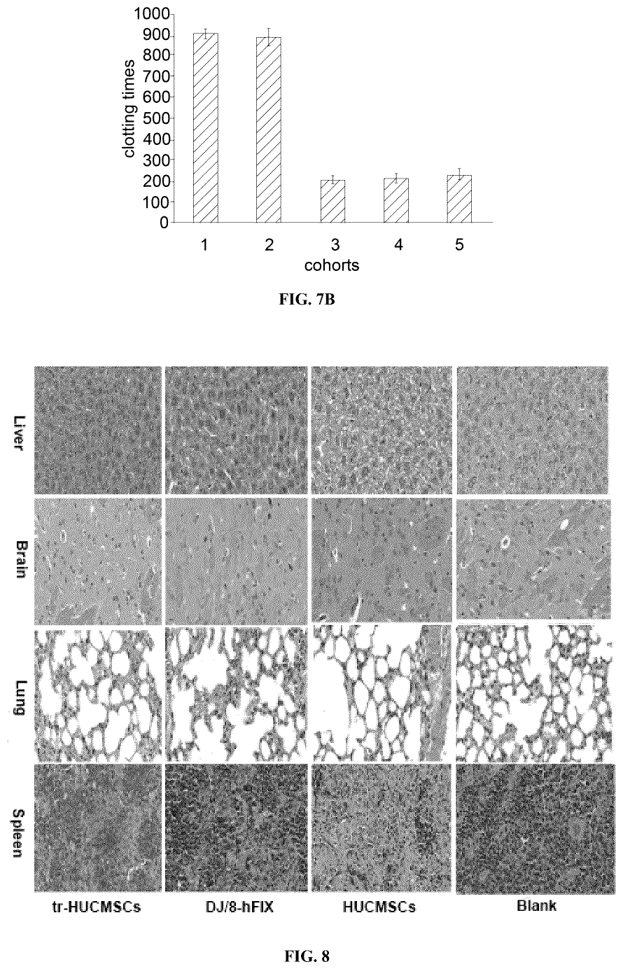

Referring to FIGS. 7A-7B, the observation of a hemostatic effect of ScAAV-DJ/8-LP1-hFIXco in treating F9-knockout mice was carried out. Three months after the tail vein of the F9-knockout mice was injected with ScAAV-DJ/8-LP1-hFIXco, tr-HUCMSCs, or HUCMSCs, the coagulation function of each group of F9-knockout mice was evaluated by cutting their tail by 1 cm. It was found that the groups directly injected with the ScAAV-DJ/8-LP1-hFIXco and tr-HUCMSCs had a significantly shorter coagulation time than that of the groups injected with the HUCMSCs and the 0.9% sodium chloride (control group).

Compared with those for the wild-type mice, the experimental results were basically the same. Histogram: 1. 0.9% sodium chloride injection group. 2. HUCMSCs (1×10³/g) injection group. 3. Wild-type (mouse in the same strain without F9 knockout). 4. tr-HUCMSCs (1×10³/g) injection group. 5. ScAAV-DJ/8-LP1-hFIXco (1×10¹¹ vg/g) injection group.

### Example 8

Referring to FIG. 8, the observation of the clonoal cell expansion after tr-HUCMSCs injection in mice was as follows. Seven months after the tail vein of NSG mice was injected with tr-HUCMSCs or untr-HUCMSCs, the mice were euthanized and their liver, spleen, brain, and lung tissues were collected to allow tissue sectioning and H&E staining. Compared with normal NSG mice, the mice injected with tr-HUCMSCs or untr-HUCMSCs exhibited no clonal cell expansion in the liver, spleen, brain, and lung tissues.

The above are merely preferred embodiments of the present disclosure, but the protection scope of the present disclosure is not limited thereto. Any equivalent replacement or modification made by a person skilled in the art according to the embodiments of the present disclosure and inventive concepts thereof within the technical scope of the present disclosure shall fall within the protection scope of the present disclosure.

What is claimed is:

1. A preparation comprising codon-optimized human coagulation factor IX (hFIXco)-transduced human umbilical cord mesenchymal stem cells (tr-HUCMSCs).

2. A method for producing the preparation comprising the tr-HUCMSCs according to claim 1, comprising the following steps:
step 1, subjecting an F9 gene to codon-optimization to obtain a hFIXco gene, loading the hFIXco gene into ScAAV-DJ/8, and then adding a human apolipo-protein hepatic control region and a human α1-antitrypsin gene promoter in front of the hFIXco gene to form a double-stranded adeno-associated virus (AAV) vector ScAAV-DJ/8-LP1-hFIXco with stably high expression and a hFIXco activity; and
step 2, transducing the ScAAV-DJ/8-LP1-hFIXco into human umbilical cord mesenchymal stem cells (HUCMSCs), mixing the ScAAV-DJ/8-LP1-hFIXco with digested HUCMSCs at a ratio of 1,000:1 to allow culture, and then collecting cells at 24 h after the mixing to obtain the preparation comprising tr-HUCMSCs.

3. The method according to claim 2, wherein, in step 1, provision of vector ScAAV8 and synthesis of ScAAV-LP1-hFIXco are entrusted to commercial companies, and the double-stranded AAV vector ScAAV-DJ/8-LP1-hFIXco is deposited in the China Center for Type Culture Collection (CCTCC) on Dec. 5, 2023, with a deposit number of CCTCC NO: V2023112.

4. The method according to claim 2, wherein the ScAAV-DJ/8-LP1-hFIXco in step 1 is assembled and amplified using an AAV-DJ/8 Helper Free Bicistronic expression system kit.

5. The method according to claim 4, wherein amplificable vectors are derived from a human embryonic kidney 293 cells transformed with simian virus 40 large T antigen (HEK293T).

6. A method for treating human hemophilia B, comprising administering to a subject in need thereof a therapeutically effective amount of the preparation comprising tr-HUCMSCs according to claim 1.

7. A method of in vivo gene therapy, comprising administering to a subject in need thereof a therapeutically effective amount of the preparation comprising tr-HUCMSCs according to claim 1.
